(19) United States
(12) Patent Application Publication
(10) Pub. No.: US 2014/0251329 A1
(43) Pub. Date: Sep. 11, 2014

(54) FRESH GAS ENTRAINMENT PORT ON THE INSPIRATORY LIMB OF A HIGH FREQUENCY OSCILLATORY VENTILATOR

(71) Applicant: Adam Whitnel Bostick, Raleigh, NC (US)

(72) Inventor: Adam Whitnel Bostick, Raleigh, NC (US)

(21) Appl. No.: 13/789,367

(22) Filed: Mar. 7, 2013

(51) Int. Cl. A61M 16/00; A61M 16/08; A61M 16/12; A61M 16/20; A61M 16/16

(52) U.S. Cl. CPC A61M 16/0057; A61M 16/208; A61M 16/16; A61M 16/12; A61M 16/0816; A61M 16/0875
USPC 128/203.12; 128/205.24

(57) ABSTRACT

An entrainment port regulates the flow of gas and/or medicine into an inspiratory limb of a high frequency oscillatory ventilator (H0FOV) to improve the oxygen content of the air entering a patient. The entrainment port is connected to an oxygen source and uses entrained oxygen therefrom to enter the inspiratory limb. Medication and/or admixtures of medication, gas and/or humidification can be introduced through the entrainment port. The flow of entrained oxygen and the mean airway pressure is regulated by a plug valve. A check valve is calibrated to a pre-defined cracking pressure to open and allow the entrained oxygen into the inspiratory limb when negative pressure is asserted in the HFOV circuit by operation of the expiratory cycle of the HFOV.

### BACKGROUND OF THE INVENTION

The present invention relates to ventilators used to aid the breathing of patients, and more particularly to High Frequency Oscillatory Ventilators (HFOVs). The present invention provides an entrainment port with a one-way check valve to reduce re-breathing of carbon dioxide and allow for introduction of fresh gas and/or aerosolized medication into the inspiratory limb of the HFOV.

Over the last decade, the HFOV has been utilized as a rescue oxygenation therapy for adults with severe acute respiratory distress syndrome (ARDS). The use of HFOVs has consistently shown short term improvement in oxygenation parameters in patients, which is attributed to the use of higher mean airway pressure (mPaw) in the HFOV. The HFOV is advantageous in that it improves on the oxygen parameters in a patient while providing reduced rates of ventilator induced lung injury when compared to conventional ventilation.

Currently, there is only one HFOV approved by the FDA for use in the United States, the Sensormedics® Model 3100B HFOV. One problem commonly associated with the 3100B HFOV is persistent hypercapnea. During the exhalation cycle, the oscillating diaphragm can generate negative pressure (e.g., sub-ambient pressure). This property of HFOV is sometimes referred to as “active exhalation”. This HFOV characteristic causes exhaled carbon dioxide (CO₂) to be entrained into the inspiratory limb of the HFOV circuit. This CO₂, if left in the inspiratory limb, can be re-breathed by the patient, contributing to increased levels of CO₂ in the patient's lungs. Such a condition can cause or contribute to hypercapnea and lead to lung injury when tidal volumes are increased to compensate for this. Although the Sensormedics 3100B HFOV is the only HFOV currently approved by the FDA for use in adult patients with ARDS, other HFOVs are coming to the market. These other HFOVs, as well as other ventilators on the market are believed to likewise have the active exhalation problem.

As can be seen, there is a need for an apparatus for HFOVs that provides adequate oxygenation and CO₂ clearance, while minimizing injurious stresses on the lung.

### SUMMARY OF THE INVENTION

In one aspect of the present invention, an entrainment port for introducing a flow of fresh gas into a high frequency oscillatory ventilator (HFOV) circuit comprises an inspiratory limb assembly; a check valve assembly connected to the inspiratory limb assembly, where the inspiratory limb assembly fluidly connects an inspiratory limb of the HFOV circuit with the check valve assembly; a check valve in the check valve assembly, the check valve regulating the flow through the check valve assembly toward the inspiratory limb assembly; a plug valve assembly fluidly connected to the check valve assembly; a plug valve operable to adjust a volume of the flow through the plug valve assembly; and a connector operable to deliver the flow into the plug valve assembly.

In another aspect of the present invention, an entrainment port for introducing a flow of fresh gas into a high frequency oscillatory ventilator (HFOV) circuit comprises an inspiratory limb assembly; a check valve assembly connected to the inspiratory limb assembly, where the inspiratory limb assembly fluidly connects an inspiratory limb of the HFOV circuit with the check valve assembly; a check valve in the check valve assembly, the check valve regulating the flow through the check valve assembly toward the inspiratory limb assembly; a plug valve assembly fluidly connected to the check valve assembly; a plug valve operable to adjust a volume of the flow through the plug valve assembly; a t-connector operable to deliver the flow into the plug valve assembly; a check wheel in the check valve assembly; and a perforated safety catch disposed between the check valve and the inspiratory limb.

In a further aspect of the present invention, a method for reducing carbon dioxide entrainment within an inspiratory limb of a high frequency oscillatory ventilator (HFOV) comprises delivering a flow through a connector, fluidly connected to a plug valve assembly having a plug valve, through a check valve and into the inspiratory limb when a negative pressure exists in the inspiratory limb; and releasing a volume of air out of an exhale port of the HFOV equal to the volume of air entrained from an entrainment port fluidly connected to the inspiratory limb, the entrainment port including the connector, the plug valve assembly and the check valve.

These and other features, aspects and advantages of the present invention will become better understood with reference to the following drawings, description and claims.

### BRIEF DESCRIPTION OF THE DRAWINGS

FIG. 1 is a perspective view of a fresh gas entrainment port, installed on an inspiratory limb of a HFOV, according to an exemplary embodiment of the present invention

FIG. 2 is a schematic view of the fresh gas entrainment port of FIG. 1, illustrating its installation in a HFOV system;

FIG. 3 is an exploded perspective view of the fresh gas entrainment port of FIG. 1; and

FIG. 4 is a cross-sectional view taken along line 4-4 of FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an entrainment port for regulating the flow of gas and/or medicine into an inspiratory limb of a HFOV to improve the oxygen content of the air entering a patient. The entrainment port is connected to an oxygen source and uses entrained oxygen therefrom to enter the inspiratory limb. Medication and/or admixtures of medication, gas and/or humidification can be introduced through the entrainment port. The flow of entrained oxygen and the mean airway pressure is regulated by a plug valve. A check valve is calibrated to a pre-defined cracking pressure to open and allow the entrained oxygen into the inspiratory limb when negative pressure is asserted in the HFOV circuit by operation of the expiratory cycle of the HFOV.

It has been discovered that the negative pressure detectable within the inspiratory limb of a HFOV, such as the Sensormedics® Model 3100B HFOV circuit, varies inversely with mean airway pressure (mPaw), which is the average pressure over one inspiration/exhalation cycle. Moreover, it has been discovered that the negative pressure detectable within the inspiratory limb of the HFOV circuit varies directly with oscillatory pressure (ΔP) within the inspiratory limb.

Specifically, through experimentation, as discussed in greater detail below, it was determined that CO₂ levels increased in the inspiratory limb when more negative pressure was generated therein, and increased with an increase in ΔP or a decrease in mPaw. It has been discovered low ΔP, high mPaw, high oscillatory frequency (Hz), high bias flow, and cuff leak placement are all factors that decreased CO₂ entrainment. CO₂ entrainment was also reduced by utilizing a higher bias flow strategy at any targeted mPaw. It has been concluded that entrainment of CO₂ is directly proportional to the amount of negative pressure generated within the inspiratory limb of the HFOV circuit.

Based on experiments and conclusions therefrom, it has been concluded that it is desirable to provide an apparatus that alleviates the problem of CO₂ entrainment. This device would reduce CO₂ re-breathing and improve blood gas values while maximizing lung protective ventilation during the use of HFOV.

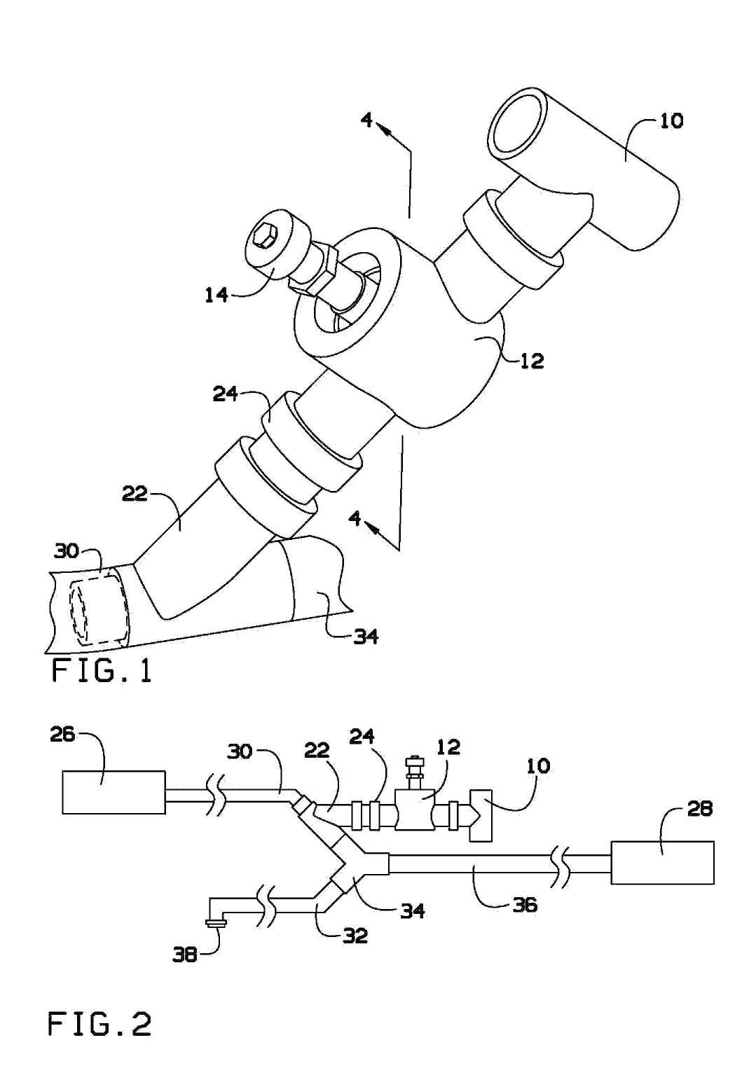

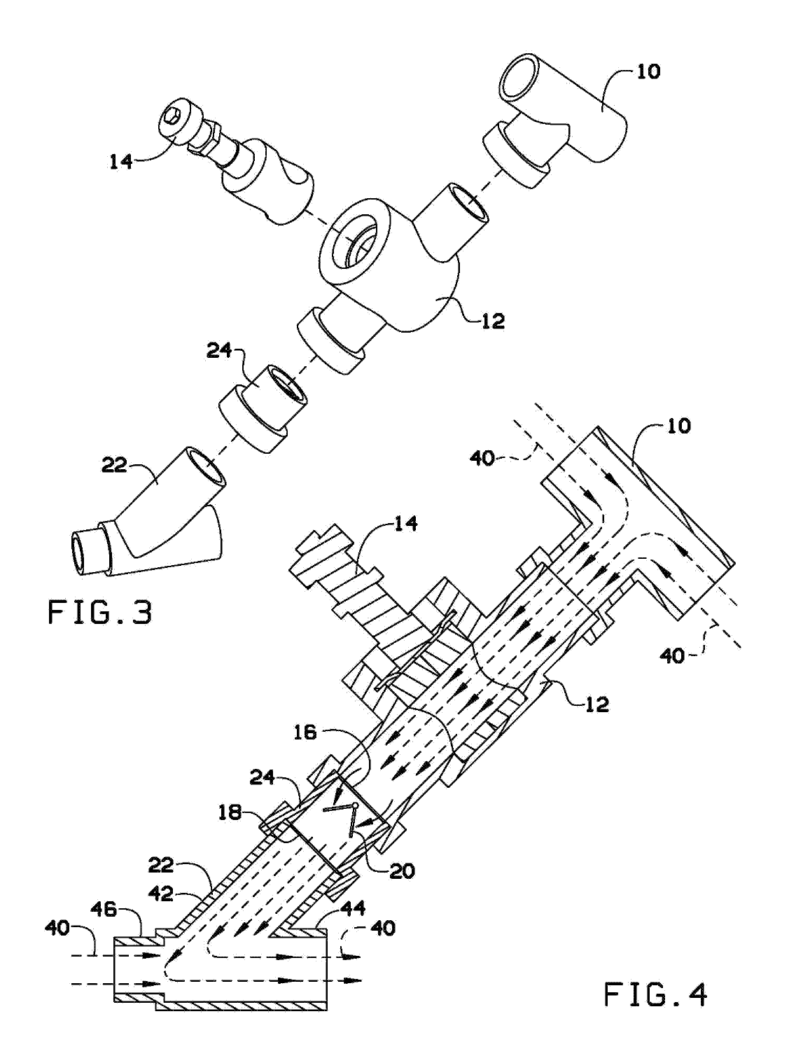

Referring now to FIGS. 1 through 4, a gas entrainment device includes a novel one way entrainment port for placement in an HFOV circuit which accomplishes the above objectives and alleviates the problem of CO₂ entrainment within the inspiratory limb of an HFOV. The entrainment port also provides a mechanism for introducing aerosolized medication to the patient via the HFOV circuit which is currently not feasible. A typical HFOV circuit has a wye 34, an inspiratory limb 30 and an expiratory limb 32. The wye 34 divides the expiratory limb 32 from the inspiratory limb 30 outside of the patient's mouth. Extending from the HFOV circuit, and particularly the wye 34, is an endotracheal tube (ETT) 36 extending into the trachea of a patient 28.

The expiratory limb 32 extends a predefined distance, and terminates in an exhale port 28 for transporting and venting exhaled air from the patient 28 out of the HFOV circuit. In a typical HFOV set up, the inspiratory limb 30 extends from the wye 34 and is connected at its distal end to the HFOV 26. A temperature port (not shown) is disposed along the wall of the inspiratory limb 30 adjacent the wye 34, and a pressure relief valve (not shown) is located along the proximal end of the inspiratory limb 30 close to the HFOV 26.

The entrainment port is designed to connect to a “T” shaped t-connector 10 at its distal end. The t-connector 10 receives a portion of, and is adjacent an adjustable plug valve assembly 12. The plug valve assembly 12 defines a passageway for the flow of entrained gas (oxygen and/or medication) from an external source (not shown). The plug valve assembly 12 houses a plug valve 14 that is adjustable to control the rate of flow of oxygen/medication through the entrainment port and to open and close the flow regulating valve when desired. The plug valve 14 regulates the mPaw by regulating the airflow through the passageway.

Downstream from the plug valve 14 is a receiving portion of the plug valve assembly that receives a check valve assembly 24 therein. The check valve assembly 24 defines a passageway substantially congruent to the passageway defined by the plug valve assembly 12. The check valve assembly 24 houses a check valve 20. The check valve assembly 24 opens toward the inspiratory limb 30 of the HFOV. Typically, the check valve 20 is a rubber check valve that is calibrated to open or crack at a desired or predefined cracking pressure. It should be understood that the check valve 20 may be calibrated to open at different pressure levels, as desired to control the mPaw. Downstream from the check valve 20 is a receiving portion of the check valve assembly 24 which receives a valve arm of an inspiratory limb assembly 22 of the entrainment port.

The inspiratory limb assembly 22 is substantially “Y” shaped and connects the one way entrainment port into the inspiratory limb 30 of the HFOV. The inspiratory limb assembly 22 comprises a valve arm and an inspiratory limb arm. The valve arm defines a passageway, and is received by receiving portion of the check valve assembly 24. The inspiratory limb arm defines a passageway substantially congruent in diameter to the diameter of the inspiratory limb 30. At its distal end, which is the end farthest from the patient, the inspiratory limb arm is tapered to slide within a coupling portion of the inspiratory limb 30. The proximal end of the inspiratory limb arm of the inspiratory limb assembly 22, which is the end closest to the patient, receives the inspiratory limb 30 therein. The passageways of the valve arm and the inspiratory limb arm converge within the inspiratory limb assembly 22 to form one passageway.

In operation, as the driving piston (not shown) of the HFOV retracts (exhalation cycle), negative pressure is generated within the inspiratory limb 30. The negative pressure opens the check valve 20 and allows for entrainment of fresh gas/medication 40 from the entrainment port. This fresh gas replaces the previously entrained CO₂ in an air neutral fashion, meaning that the same volume of gas entrained from the entrainment port is released from the exhale port 38 negating any substantial changes in mPaw. At the end of the exhalation cycle the driving piston of the HFOV advances, generating positive pressure within the inspiratory limb 30, and closes the check valve 20 of the entrainment port during the inhalation cycle. By allowing fresh gas to be entrained from the entrainment port into the inspiratory limb 30 during the exhalation cycle of the HFOV, as opposed to the CO₂ from the patient, total functional dead space is thus reduced. This prevents re-breathing of CO₂, allowing for improvement in blood CO₂ clearance. The entrained gas from the entrainment port can be any admixture of oxygen, humidification, or medication that is passing by the flow regulating valve. The delivery of gas to the entrainment port is a standard T-piece with corrugated tubing.

### THE EXPERIMENTAL DESIGN

Efforts in the present invention are the first known detection and study the problem of CO₂ entrainment into the inspiratory limb of an HFOV, and specifically within the Sensormedics® Model 3100B HFOV.

An experimental design was set up in which a 3100B HFOV was interfaced to a cuffed endotracheal tube (ETT) and connected to a test lung. Negative pressure changes within the circuit's inspiratory limb were measured while HFOV settings were manipulated. Retrograde CO₂ entrainment was then evaluated by insufflating CO₂ into the test lung, achieving 40 mmHg at the carina. CO₂ entrainment within the inspiratory limb was measured at incremental distances from the wye. The HFOV settings and cuff leak from an ETT cuff were varied to assess their effect on CO₂ entrainment. Control experiments were conducted using a conventional ventilator. Test lung results were then validated on a large hypercapnic swine model (not shown).

The Test Lung Experiment. HFOV Inspiratory Limb Pressure Measurements

The 3100B HFOV (C are Fusion, San Diego, Calif.) was set up in line with a cuffed 8.0 mm ETT positioned within an artificial trachea. The artificial trachea was attached to a test lung set with a compliance of 0.02 L/cm H₂O (Michigan Instruments Test Lung Model 5600i, Grand Rapids, Mich.). A pressure transducer (TruWave, Edwards Lifesciences, Irvine, Calif.) with a maximum frequency response of 200 Hz was adapted to fit the temperature port on the inspiratory limb of the HFOV circuit located 1 inch from the wye (wye-1″)). The transducer was leveled with the temperature port and calibrated. Pressure tracings were displayed on a Solar 8000i hemodynamic monitor (General Electric, Fairfield, Conn.). Peak positive and negative pressure values were identified with the pressure display cursor and recorded.

Peak negative pressure (e.g. during exhalation cycle of the piston) was measured while adjusting mPaw from 50 cm H₂O to 20 cm H₂O. Other HFOV parameters were as follows: ΔP 90 cm H₂O, bias flow 30 LPM, I-time 33%, Hz 7. Pressure tracings were also measured while adjusting ΔP from 120 cm H₂O to 30 cm H₂O. HFOV parameters for ΔP experiments were as follows: mPaw 34 cm H₂O, bias flow 30 LPM, I-time 33%, Hz 7.

### CO₂ Entrainment During HFOV in Mechanical Lung Model

The 3100B HFOV and mechanical test lung were set up as previously described (with exception of the pressure transducer). An RGM 5250 gas analyzing line (Datex-Ohmeda, Madison, Wis.) was inserted at a point 30 inches from the wye (wye-30″) within the inspiratory limb of the HFOV circuit and then positioned at the carina 18. CO₂ was insufflated (7% CO₂ H-tank 30) into the test lung circuit at a flow rate of 0.5 LPM to attain 40 mm Hg CO₂ at the carina 18. The settings of the 3100B HFOV during this calibration period were: mPaw 34 cm H₂O, bias flow 30 LPM, I-time 33%, Hz 7, ΔP 90 cm H₂O, FiO₂ 0.21. These settings were used to calibrate CO₂ insufflation for each of the subsequent experiments. Gas sampling at the carina was performed prior to all experiments, and at regular intervals during experiments, to verify that carinal CO₂ remained at 40 mm Hg. All mechanical lung experiments were performed in duplicate and the averaged data reported.

The gas analyzer was withdrawn back from the carina into the inspiratory limb of the HFOV circuit to a maximum of wye-35″ confirming the presence of retrograde CO₂ entrainment. After confirming CO₂ was detectable within the inspiratory limb, the gas analyzer was placed at the wye-20″ position and the different parameters (mPaw, bias flow, Hz, ΔP) of the 3100B HFOV were independently manipulated to assess each setting's effect on CO₂ entrainment. Evaluation of CO₂ entrainment was also performed while simultaneously increasing bias flow and manipulating the mean pressure adjustment to maintain a constant mPaw of 34 cm H₂O. All experiments were performed with and without a cuff leak.

There are two different techniques available to introduce a 5 cm H₂O ETT cuff leak. One method is to increase the bias flow to attain an mPaw 5 cm H₂O higher than the current targeted mPaw and then deflate the ETT cuff until the mPaw reaches the original value. The other method is to increase the mean pressure adjustment and deflate the ETT cuff in a similar fashion as just described (with a constant bias flow). Both of these cuff leak methods were evaluated by measuring entrained CO₂ at the wye-20″ position.

### CO₂ Entrainment During Conventional Ventilation in Mechanical Lung Model

Conventional ventilation experiments with a Servo-i ventilator (Maquet, Wayne, N.J.) (not shown) using continuous mandatory ventilation (CMV) and Bi-Vent modes were performed. This experiment assessed for possible retrograde CO₂ entrainment during conventional ventilation and served as a control for the experimental test lung setup. CO₂ insufflation was performed during these control experiments by two different methods: 1) 40 mm Hg CO₂ (measured at end exhalation) was attained at the carina prior to manipulating the ventilator parameters (CO₂ flow of 0.1 LPM), and 2) CO₂ flow of 0.5 LPM to match the same flow as used during the HFOV experiments (producing a carinal CO₂ of >107 mm Hg which is above the limit of the gas analyzer). Test lung compliance was set at 0.02 L/cm H₂O.

The Servo-i ventilator was placed in CMV mode with the parameters set as follows: V_{T} 420 mL (simulating 70 kg patient at 6 mL/kg), positive end expiratory pressure (PEEP) 10 cm H₂O, respiratory rate (RR) 15 breaths per minute (bpm), flow trigger 3 LPM, FiO₂ 0.4, I:E 1:3. With insufflated CO₂ flow at 0.1 LPM, the gas analyzer was withdrawn back into the inspiratory limb and measurements of CO₂ entrainment were obtained at incremental distances. The analyzer was then positioned at wye-3″ and the effect on CO₂ entrainment was assessed while manipulating the different ventilator parameters independently as follows: PEEP 5-25 cm H₂O, RR 10-30 bpm, V_{T} 100-700 mL, flow trigger 3 LPM and pressure trigger −2 cm H₂O. The CO₂ flow was then increased to 0.5 LPM and the identical measurements were performed.

The Servo-i ventilator was then place in Bi-Vent mode with baseline settings as follows: P_{high} 30 cm H₂O, P_{low} 5 cm H₂O, I:E=4:1, flow trigger 5 LPM, FiO₂ 0.4. The gas analyzer was placed at wye-3″ and effects on CO₂ entrainment were assessed while manipulating the different parameters independently as follows: P_{high} 20-34 cm H₂O, P_{low} 0-20 cm H₂O, I:E 1:5-5:1. CO₂ flow rates of 0.1 LPM and 0.5 LPM were utilized as done during CMV experiments.

### CO₂ Entrainment During HFOV in Swine Model

Once retrograde CO₂ entrainment during HFOV was characterized with the mechanical lung model, a feasibility study was performed on a 75 kg swine (Sus scrofa) (not shown) to determine if similar phenomena occurred in vivo. The use of this swine was approved by the Wilford Hall Medical Center IACUC board and was performed during an ongoing training protocol. The swine was induced with isoflurane and intubated with a 7.0 mm ETT (not shown). A fentanyl infusion was then used to continue analgesia-sedation as the swine was transitioned to a 3100B HFOV. In a similar fashion to the test lung experiment (without insufflated CO₂), the gas analyzing line was inserted into the inspiratory limb of the circuit and entrained CO₂ was measured at incremental distances from the wye. The initial HFOV settings were identical to those used during the calibration period of the mechanical test lung. The swine was briefly hypoventilated between experiments by increasing Hz for 10 seconds to achieve mild hypercapnea (PₐCO₂ 52.5-63.8 mm Hg). Once retrograde CO₂ entrainment was confirmed within the inspiratory limb, the gas sampling line was placed at the wye-10″ position and the HFOV parameters were independently manipulated (mPaw, bias flow, Hz, ΔP) to assess their effects on CO₂ entrainment. The effect of a 5 cm H₂O ETT cuff leak was also assessed.

The Results: HFOV Inspiratory Limb Pressure Measurements

Negative pressure was readily measured within the inspiratory limb of the HFOV circuit and varied inversely with mPaw and directly with ΔP. More negative pressure was produced when the mPaw was reduced from 50 cm H₂O to 20 cm H₂O at a fixed ΔP of 90 cm H₂O. In contrast, negative pressure became undetectable when ΔP was reduced from 120 cm H₂O to 60 cm H₂O at a constant mPaw 34 cm H₂O. An increase in retrograde CO₂ entrainment occurred when more negative pressure was generated (during exhalation cycle of piston) within the inspiratory limb of the circuit.

### The Results: CO₂ Entrainment During HFOV in Mechanical Lung Model

When the oscillating piston (not shown) of the HFOV was inactivated, no CO₂ was detected within the entire inspiratory limb of the circuit. With the piston turned on, CO₂ became readily detectable within the inspiratory limb of the 3100B HFOV circuit. Without a cuff leak, retrograde CO₂ entrainment was 22 mm Hg at wye-5″. Entrained CO₂ steadily decreased 1 mm Hg for every 1-2 inches from the wye, dissipating to 0 mm Hg at wye-35″.

The effect of increasing mPaw from 20-50 cm H₂O using either bias flow or mean pressure adjustment reduced retrograde CO₂ entrainment. Increasing mPaw by adjusting bias flow, however, had a more significant effect on reducing CO₂ entrainment when compared to utilizing the mean pressure adjustment. The effect of a 5 cm H₂O cuff leak lowered the amount of CO₂ entrainment in both cases, and the trends were comparable to those seen without a cuff leak.

CO₂ entrainment when increasing bias flow incrementally from 20-60 LPM while maintaining a constant mPaw of 34 cm H₂O, in the absence of a cuff leak, resulted in 14 mm Hg of CO₂ was detected at a bias flow of 20 LPM and decreased to 7 mm Hg at a bias flow of 60 LPM. A 5 cm H₂O cuff leak further reduced CO₂ entrainment once bias flow was increased above 30 LPM.

By increasing ΔP incrementally from 10-120 cm H₂O, in the absence of a cuff leak, CO₂ entrainment became detectable at 6 mm Hg once ΔP reached 70 cm H₂O. Entrained CO₂ increased to 13 mm Hg at a ΔP of 120 cm H₂O. A 5 cm H₂O cuff leak reduced retrograde CO₂ entrainment to 1 mm Hg at a ΔP of 70 cm H₂O and 9 mm Hg at a ΔP of 120 cm H₂O.

Increasing from 2-15 Hz, incrementally, resulted in CO₂ entrainment being decreased as Hz was increased. In the presence of a cuff leak, retrograde CO₂ entrainment was further reduced at frequencies less than 10.

The two methods of placing an ETT cuff leak and their effect on retrograde CO₂ entrainment were analyzed. Prior to performing an ETT cuff leak, 11 mm Hg CO₂ was measured at the wye-20″ position. When bias flow was used to produce a cuff leak there was 7 mm Hg of CO₂ entrainment. When the mean pressure adjustment was used to produce a cuff leak, CO₂ entrainment increased to 9 mm Hg.

### The Results: CO₂ Entrainment During Conventional Ventilation in Mechanical Lung Model

With the Servo-i ventilator (not shown) at baseline CMV settings and insufflated CO₂ at 0.1 LPM, there was 8 mm Hg CO₂ detectable at wye-1″. Entrained CO₂ dissipated to 0 mm Hg at wye-2″. There was no detectable CO₂ entrainment, measured at wye-3″, when the different ventilator parameters were manipulated.

At baseline CMV settings with insufflated CO₂ flow at 0.5 LPM, the carinal CO₂ was >107 mm Hg (above the limit of the gas analyzer). Entrained CO₂ dissipated to 0 mm Hg at the wye-3″ position. Again, there was no detectable CO₂ entrainment at wye-3″ when the different ventilator parameters were manipulated.

With the Servo-i ventilator on baseline Bi-Vent settings and CO₂ insufflated at 0.1 LPM, there was 8 mm Hg CO₂ detectable at wye-1″. Entrained CO₂ dissipated to 0 mm Hg at wye-2″. No detectable CO₂ entrainment occurred when the different ventilator parameters were manipulated.

At baseline Bi-Vent settings and the insufflated CO₂ flow at 0.5 LPM, the carinal CO₂ was >107 mm Hg. Entrained CO₂ dissipated to 0 mm Hg at wye-3″. Again, there was no detectable CO₂ entrainment at wye-3″ when the different ventilator parameters were manipulated.

### The Results: CO₂ Entrainment During HFOV in Swine Model

With the swine (not shown) on the HFOV (settings: mPaw 34 cm H₂O, bias flow 30 LPM, I-time 33%, Hz 7, ΔP 90 cm H₂O, FiO₂ 1.0) and the ETT cuff 22 maximally inflated, 10 mm Hg of CO₂ was detectable within the wye. The gas analyzer was withdrawn into the inspiratory limb revealing 10 mm Hg of CO₂ at wye-5″ which dissipated to 3 mm Hg at wye-30″. Despite maximal cuff inflation, 10 mm Hg CO₂ was persistently detectable within the swine's oropharynx. A 5 cm H₂O cuff leak (in addition to the persistent leak with cuff maximally inflated) was then placed which reduced CO₂ entrainment to 9 mm Hg within the wye, 6 mm Hg at wye-5″ and 2 mm Hg at wye-30″.

The effect of increasing mPaw by using the mean pressure adjustment, in the presence of a cuff leak was examined. Entrained CO₂ was reduced from 8 mm Hg to 5 mm Hg when mPaw was increased from 24 cm H₂O to 34 cm H₂O.

The effect of ΔP on CO₂ entrainment in the swine was examined. With a cuff leak in place, entrained CO₂ at wye-10″ was increased from 2 mm Hg to 6 mm Hg when increasing ΔP from 60 cm H₂O to 90 cm H₂O. The effect of adjusting Hz, with a 5 cm H₂O cuff leak in place, revealed 10 mm Hg of entrained CO₂ at 3 Hz which decreased to 1 mm Hg at 15 Hz.

These findings demonstrate that carbon dioxide is readily detectable within the inspiratory limb of the Sensormedics 3100B HFOV in both the mechanical lung and swine models. Retrograde CO₂ entrainment was identified as far back as 30 inches from the wye in both models, suggesting functional total dead space may extend well into the inspiratory limb of the circuit. This is a unique characteristic of the 3100B HFOV that has not previously been reported. In contrast, CO₂ rebreathing has been identified in non-invasive ventilation such as single circuit BiPAP systems (not shown).

This data demonstrate that retrograde CO₂ entrainment during HFOV is proportional to the amount of negative pressure generated within the inspiratory limb of the circuit and varies directly with ΔP and inversely with mPaw (e.g. more negative pressure occurs when a higher ΔP is combined with a lower mPaw). During HFOV, retrograde CO₂ entrainment is reduced by the following: increasing mPaw, decreasing ΔP, placement of a cuff leak, increasing Hz, and increasing bias flow at any targeted mPaw.

A limitation to the mechanical lung model experiments included assuring CO₂ insufflation was comparable between the HFOV and conventional ventilator control experiments. This was reconciled by insufflating CO₂ during conventional ventilation at 0.1 LPM to achieve 40 mm Hg at the carina (identical partial pressure as during HFOV experiment), as well as insufflating at 0.5 LPM (identical flow as during HFOV experiment) which created levels of CO₂ at the carina above the limit of the RGM 5250 gas analyzer (>107 mm Hg). In neither case was retrograde CO₂ entrainment detected beyond wye-3″. Furthermore, no CO₂ was detected within the inspiratory limb of the 3100B HFOV circuit when the piston (not shown) was inactivated (e.g. no negative pressure within the inspiratory limb 16 of the circuit).

There were quantitative differences in the amount of CO₂ entrainment between the mechanical lung model and swine. This finding was attributable to a persistent cuff leak in the swine despite maximal ETT cuff inflation (oropharyngeal CO₂ of 10 mm Hg). Additionally, there are inherent differences in CO₂ production with a live animal. Despite the lower levels of entrained CO₂ measured in the swine, the trends in CO₂ entrainment with manipulations of HFOV parameters were similar when compared to the mechanical test lung.

### The Solution: The Entrainment Port

Referring again to FIGS. 1 through 4, according to aspects of the present invention, an entrainment port can provide a solution to the problem of CO₂ entrainment within the inspiratory limb of the HFOV. Specifically, the entrainment port of the present invention is designed to be integrated within the inspiratory limb 30 of the HFOV circuit. The HFOV circuit comprises the Sensormedics® 3100B HFOV 26 connected to the inspiratory limb 30. The wye 34 joins the inspiratory limb 30 and the expiratory limb 32. The ETT 36 extends from wye 34 into the trachea (not shown) of the patient 28.

The entrainment port connects to the t-connector 10. T-connector 10 defines a fresh gas source pathway to which a gas source (not shown) is attached. T-connector 10 is of the type of connectors typically used in medical applications to connect to gas sources such as oxygen sources. The primary flow of oxygen is indicated by the arrows 40 through the pathway. A first pathway of the t-connector 10 is perpendicular to a second pathway and provides a passageway for oxygen entrained from the main flow through the first pathway to flow, as indicated by the arrows 40 leading from the first pathway into the second pathway. At the lower end of the second pathway, the t-connector 10 comprises a receiving connector for receiving the adjacent plug valve assembly 12 by, for example, receiving a sidewall of the plug valve assembly 12 therein such that the sidewall abuts the second pathway of the t-connector 10.

The plug valve assembly 12 defines a pathway which is substantially the same diameter and adjoins with the second pathway of the t-connector 10. The plug valve assembly 12 houses the plug valve 14 approximately along the midpoint thereof. The plug valve 14 is typically a ball valve that rotates within the pathway of the plug valve assembly 12 to regulate the flow of entrained gas. However, the plug valve 14 can be a needle valve, or any suitable valve that will regulate airflow therethrough by either rotating within the pathway, or sliding in and out of the pathway, for example, to adjust the cross sectional diameter of the pathway. By controlling the airflow through the pathway, the plug valve 14 regulates the mPaw of the entrainment port. In some embodiments, the plug valve assembly 12 includes a receiving connector with shoulders. The receiving connector is substantially the same as receiving connector of the t-connector 10, for example.

The check valve assembly 24 defines a pathway, which is substantially the same diameter, and adjoins with the pathway of the plug valve assembly 12. The check valve assembly 24 houses a check wheel 16 which has a central fulcrum to which the check valve 20 is attached, preventing the check valve 20 from opening in the wrong direction. Thus, the check valve 20 is designed as a one-way valve. In an exemplary embodiment, the check valve 20 is a rubber diaphragm check valve as commonly known in the art, but it should be understood that any fast acting one-way check valve, including but not limited to a Reid valve or butterfly valve, could be used. The check valve 20 is oriented to open towards the inspiratory limb assembly 22 and close toward the plug valve assembly 12. It should be understood that the check valve 20 should have a predefined cracking pressure such that it meets the desired cracking pressure to open at the appropriate variable negative pressure asserted on the entrainment port. The check valve assembly 24 can be connected to the inspiratory limb assembly 22 in various manners as known in the art.

The inspiratory limb assembly 22 is substantially “Y” shaped and comprises a valve arm 42 and an inspiratory limb arm 44. The valve arm 42 defines a pathway which is substantially the same diameter, and adjoins the pathway of the check valve assembly 22. The inspiratory limb arm 44 defines an inspiratory limb pathway which converges with the valve arm pathway. A perforated safety catch 18 can be disposed within the valve arm 42, spanning the diameter thereof. The perforated safety catch 18 provides a safety catch in the event that the check valve 20 or the check wheel 16 are dislodged, preventing entry into the airway of the patient 28.

The end of the inspiratory limb arm 44 closest to the patient 28 receives a portion of the inspiratory limb 30 within its pathway, and adjoins thereto. The opposite end of the inspiratory limb arm 44 forms a neck 46 which is slightly decreased in diameter from the rest of the inspiratory limb arm 44. The neck 46 can be inserted into a collar of the inspiratory limb 30, thus integrating the entrainment port into the inspiratory limb 30 of the HFOV circuit. As shown, the entrainment port is disposed adjacent or in close proximity to the wye 34.

The HFOV 26 comprises a driving piston (not shown) which drives in an advancing direction (not shown) during the inhalation cycle and retracts in a retracting direction (not shown) opposite the first direction during the exhalation cycle. During the inhalation cycle, the piston advances toward the inspiratory limb 30, generating positive pressure. During the exhalation cycle, the piston retracts, generating negative pressure within the inspiratory limb 30. Without the entrainment port of the present invention, some breathed gas is entrained backwards into the inspiratory limb 30 during the expiratory cycle due to the negative pressure within the inspiratory limb 30 caused by the retraction of the HFOV piston (not shown). By adding the entrainment port of the present invention, connected to a fresh gas source, fresh air enters the inspiratory limb 30 close to wye 34.

As discussed above, as the driving piston of the HFOV 26 retracts during the exhalation cycle, the negative pressure opens the check valve 20 and allows for entrainment of fresh gas and/or medication from the entrainment port. This fresh gas replaces the previously entrained CO₂ within the inspiratory limb 30 in an air neutral fashion, meaning that the same volume of fresh gas entrained from the entrainment port is released from the exhale port 38 negating any substantial changes in mPaw.

At the end of the exhalation cycle the driving piston of the HFOV 26 advances, generating positive pressure within the inspiratory limb 30, closing the check valve 20 of the entrainment port during the inhalation cycle. By allowing fresh gas to be entrained from the entrainment port into the inspiratory limb 30 during the exhalation cycle of the HFOV 26, rebreathing of CO₂ previously entrained into the inspiratory limb 30 is alleviated, allowing for improvement in blood CO₂ clearance. Again, although described as fresh gas, the entrained gas from the entrainment port can be any admixture of oxygen, humidification, and/or medication that is passing through the entrainment port.

Moreover, it should be understood that the t-connector, 10, plug valve assembly 12, check valve assembly 24 and inspiratory limb assembly 22 are connected in such a way as to form substantially air-tight connection. In one example, these components are made from corrugated tubing. However, any other suitable material may be used. Moreover, although described as comprising component assemblies 10, 12, 24 and 22, the entrainment port could be manufactured as a single piece port, thus eliminating the need for the connectors between the components.

It should be understood, of course, that the foregoing relates to exemplary embodiments of the invention and that modifications may be made without departing from the spirit and scope of the invention as set forth in the following claims.

What is claimed is:

1. An entrainment port for introducing a flow into a high frequency oscillatory ventilator (HFOV) circuit, the entrainment port comprising:
an inspiratory limb assembly;
a check valve assembly connected to the inspiratory limb assembly, where the inspiratory limb assembly fluidly connects an inspiratory limb of the HFOV circuit with the check valve assembly;
a check valve in the check valve assembly, the check valve regulating the flow through the check valve assembly toward the inspiratory limb assembly;
a plug valve assembly fluidly connected to the check valve assembly;
a plug valve operable to adjust a volume of the flow through the plug valve assembly; and
a connector operable to deliver the flow into the plug valve assembly.

2. The entrainment port of claim 1, wherein the flow is includes at least one of oxygen, medication and humidification.

3. The entrainment port of claim 1, wherein the connector is a t-connector.

4. The entrainment port of claim 1, further comprising a check wheel in the check valve assembly.

5. The entrainment port of claim 1, further comprising a perforated safety catch disposed between the check valve and the inspiratory limb.

6. An entrainment port for introducing a flow into a high frequency oscillatory ventilator (HFOV) circuit, the entrainment port comprising:
an inspiratory limb assembly;
a check valve assembly connected to the inspiratory limb assembly, where the inspiratory limb assembly fluidly connects an inspiratory limb of the HFOV circuit with the check valve assembly;
a check valve in the check valve assembly, the check valve regulating the flow through the check valve assembly toward the inspiratory limb assembly;
a plug valve assembly fluidly connected to the check valve assembly;
a plug valve operable to adjust a volume of the flow through the plug valve assembly;
a t-connector operable to deliver the flow into the plug valve assembly;
a check wheel in the check valve assembly; and
a perforated safety catch disposed between the check valve and the inspiratory limb.

7. The entrainment port of claim 6, wherein the flow is includes at least one of oxygen, medication and humidification.

8. A method for reducing carbon dioxide entrainment within an inspiratory limb of a high frequency oscillatory ventilator (HFOV), the method comprising:
delivering a flow through a connector, fluidly connected to a plug valve assembly having a plug valve, through a check valve and into the inspiratory limb when a negative pressure exists in the inspiratory limb; and
releasing a volume of air out of an exhale port of the HFOV equal to the volume of air entrained from an entrainment port fluidly connected to the inspiratory limb, the entrainment port including the connector, the plug valve assembly and the check valve.

9. The method of claim 8, wherein the negative pressure is formed during an exhalation cycle of the HFOV.

10. The method of claim 8 wherein the flow includes one or more admixtures of oxygen, humidification and medication.
